(19) United States
(12) Patent Application Publication
(10) Pub. No.: US 2006/0083697 A1
(43) Pub. Date: Apr. 20, 2006

(54) Topical skin lightening cream

(76) Inventors: Anthony Vu Huynh, El Centro, CA (US); Tien-Li Lee, La Jolla, CA (US)

Correspondence Address: DAVID B. WALLER & ASSOCIATES, 5677 OBERLIN DRIVE, SUITE 214, SAN DIEGO, CA, 92121, US

(21) Appl. No.: 10/966,004

(22) Filed: Oct. 15, 2004

(51) Int. Cl. A61K 8/63; A61K 8/34

(52) U.S. Cl. 424/59; 424/62

(57) ABSTRACT

The present invention is a novel topical composition for the treatment of non-congenital dermal blemishes comprising, hydroquinone wherein the concentration of hydroquinone is not less than 2 percent and not more than 12 percent, tretinoin wherein the concentration of tretinoin is not less than 0.01 percent and not more than 0.10 percent, a steroid wherein the steroid is selected from class 5, 6 or 7 steroids; and sunscreen having a sun protection factor of not less than 15 and not more than 50.

### TECHNICAL FIELD

The present invention is a novel topical cream formulation used for reducing the appearance of dermatological blemishes. More particularly, to reduce or eliminate the appearance of melasma, solar lentigo, post-inflammatory hyperpigmentation and other non-congenital hyperpigmentation diseases.

### BACKGROUND OF THE INVENTION

A variety of skin lightening products are currently available on the market. Some of these employ a single active ingredient while others employ a formulation of two or more additive or synergistic compounds. Unfortunately the effect of a single ingredient is usually limited and each known formula has notable disadvantages and often introduces unintentional side effects concomitant with treatment of the primary condition of undesired pigmentation.

Some currently available products include Triluma™ which contains 4% hydroquinone, tretinoin and a fluorinated steroid, U.S. Pat. No. 6,699,464 describes a composition containing hydroquinone, rumex extract and sunscreen cream sun protection factor 15, Claripel™ which contains 4% hydroquinone and sunscreen cream sun protection factor 15 (SPF 15) and Glyquin™ which contains 4% hydroquinone, glycolic acid and sunscreen cream SPF 15. For each of these treatments, there are significant deficiencies that may be understood by the action and interaction of each component. Hydroquinone alone is effective in reducing pigment. However, it has only limited efficacy at 4 percent concentration and can cause skin inflammation and subjective irritation for the user. In addition, application can cause increased sun sensitivity and can lead to sunburn and possible darkening of the skin.

Tretinoin is an effective treatment for both wrinkles and skin pigmentation but is also known to cause skin sensitivity that can lead to skin darkening. Sunscreen may be used to protect from sun damage but is not effective in treating inflammation that often accompanies application of hydroquinone or tretinoin.

Steroids can be used to counteract the inflammatory effect of hydroquinone and tretinoin, however, if the strength of the steroid is too great such as the case with using fluorinated steroids there is a risk of developing telangiectasia.

Consequently there is a need for a topical skin lightening cream that provides effective skin lightening capabilities and does not cause significant inflammation, irritation, or sun sensitivity of the skin following application.

### SUMMARY OF THE INVENTION

The present invention provides a topical skin lightening cream for the treatment of non-congenital dermal blemishes comprising, hydroquinone, tretinoin, a low potency steroid and sunscreen. In one aspect of the present invention the concentration of hydroquinone is not less than 2 percent and not more than 12 percent, preferably not less than 4 percent and not more than 10 percent and most preferably 8 percent. The concentration of tretinoin is not less than 0.01 percent and not more than 0.10 percent, preferably not less than 0.03 percent and not more than 0.08 percent and most preferably 0.05 percent. The steroid is a low potency steroid selected from class 5, 6 or 7 steroids. Preferably the steroid is hydrocortisone or triamcilinone. When hydrocortisone is the steroid of choice its concentration is preferably not less than 0.5 percent and not more than 10.0 percent, preferably not less than 1.0 percent and not more than 5.0 percent, most preferably 2.5 percent. When the steroid is triamcilinone it is preferably unfluorinated triamcilinone at a concentration not less than 0.01 percent and not more than 0.1 percent. Preferably not less than 0.025 percent and not more than 0.05 percent most preferably 0.05 percent. The sunscreen preferably has a sun protection factor of not less than 15 and not more than 50.

In another embodiment of this aspect of the invention the topical skin lightening cream further comprises an excipient. Preferably the excipient is metabisulfite.

In yet another embodiment the topical skin lightening cream may further comprise glycolic acid at a concentration not less than 5.0 percent and not more than 15.0 percent. Preferably the glycolic acid is not less than 8.0 percent and not more than 12.0 percent. Most preferably the concentration of glycolic acid is 10.0 percent.

In another aspect of the present invention a topical skin lightening cream is provided that is less potent and may be provided to consumers over the counter. This topical skin lightening cream comprises hydroquinone, retinol, a low potency steroid and sunscreen. Preferably the concentration of hydroquinone is not more than 2 percent; the concentration of retinol is not more than 0.5 percent; the steroid is a class 5 steroid preferably hydrocortisone of a concentration of about 1.0 percent; and the sunscreen has a sun protection factor of not less than 15 and not more than 50.

In one embodiment of this aspect of the invention the topical skin lightening cream further comprising glycolic acid at a concentration not more than about 20 percent.

In other embodiments of these aspects of the present invention the topical skin lightening cream may further comprise, one or more antioxidants, one or more excipients, one or more preservatives, one or more emulsifiers, and/or one or more emollients.

### DETAILED DESCRIPTION

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety.

The present invention provides a novel topical composition for the treatment of non-congenital dermal blemishes, such as melasma, a type of hyperpigmentation that appears as dark blotchy clusters on the face often caused by hormonal fluctuations in pregnancy, birth control pills or hormone replacement therapy; solar lentigo, dark spots that result from exposure to the sun; and post-inflammatory hyperpigmentation, the dark discoloration that sometimes appears after acne heals or with trauma. In one aspect of the present invention the composition comprises hydroquinone, tretinoin, sunscreen and a low potency steroid. In a preferred embodiment the composition comprises not less than about 2 percent and not more than about 12 percent hydroquinone, preferably not less than about 4 percent and not more than about 10 percent and most preferably about 8 percent.

In another embodiment the composition comprises not less than about 0.01 percent and not more than about 0.10 percent tretinoin, preferably not less than about 0.03 percent and not more than about 0.08 percent and most preferably about 0.05 percent.

In yet another embodiment of the invention the sunscreen cream preferably has a SPF of not less than about 15 and not more than about 50. Each individual patient may experience differing sun light sensitivity consequently those having a more violent reaction may desire a higher SPF sunscreen cream then those with a more mild reaction who might prefer a lower SPF sunscreen cream. The compositions of the present invention include sunscreens which in combination with other ingredients provide a composition which has a SPF of at least 15. To provide a formula with a SPF value of not less than 15 usually requires the use of more than a single UVB sunscreen. Suitable UVB sunscreens for use in the compositions of the present invention include avobenzone, octyl methoxycinnamate (also known as octinoxate), oxybenzone and octocrylene. Avobenzone additionally functions as the preferred UVA sunscreen, however it is not measured as part of the SPF value. The amount of octyl methoxycinnamate, octocrylene, oxybenzone and/or avobenzone in a given formulation will vary depending on the sun protection factor desired. Preferably a higher sun protection factor such as SPF 50 is desirable.

In still another embodiment the low potency steroid is preferably selected from class 5, 6 or 7 steroids. Because each patient is unique they may experience different inflammatory responses to the hydroquinone and tretinoin depending on the concentration provided in the composition. Consequently, those having a stronger inflammatory response may desire an increased potency steroid such as a class 5 steroid or an increased concentration of a lower class steroid. A class 5 or lower steroid would be of sufficient strength to minimize any inflammatory effects from higher concentrations of hydroquinone but not cause undesired adverse effects such as telangiectasia. Correspondingly those individuals with a mild inflammatory response may desire a lower potency steroid such as a class 7 steroid or a lower concentration of the steroid. In a preferred composition the low potency steroid may be hydrocortisone or triamcilinone. When the low potency steroid is hydrocortisone cream it is preferably provided in a concentration not less than about 0.5 percent and not more than about 10.0 percent, particularly preferred concentrations are not less than 1.0 percent to not more than 5.0 percent and most preferably about 2.5 percent.

When the low potency steroid is triamcilinone it is preferably not fluorinated and of a concentration not less than about 0.01 percent and not more than about 0.1 percent, particularly preferred concentrations are not less than about 0.025 percent to not more than about 0.05 percent and most preferably about 0.05 percent.

The composition of the present invention may further comprise an excipient that may aid in the application of the formulation, effective uptake of the formulation, preserving the formulation or any combination thereof. A variety of excipients are available commercially and may be utilized in the formulation of the present invention. In a preferred embodiment the excipient is metabisulfite.

Emulsifiers and emollients are used to provide a suitable texture and impart the characteristics of a vanishing cream. Any suitable emollients and emulsifiers can be used in the present composition. Examples of suitable emulsifiers/emollients include, for example: ceteareth-20, cetostearyl alcohol, diethylaminethyl stearate, glyceryl dilaurate, glyceryl monostearate, glyceryl stearate, PEG-100 stearate, octyldodecyl stearoyl stearate, polysorbate 80, quaternium-26, stearyl alcohol. The emulsifiers and emollients may generally comprise from about 20 percent to about 50 percent by weight of the formulation. Other moisturizers that may be included in the formulation include for example sodium PCA, dimethicone, cyclomethicone, propylene glycol and polysiloxane derivatives.

The composition of the present invention can also include antioxidants which enhance the dermatologically useful effect of the formulation. Example of suitable antioxidants include vitamin E, sodium metabisulfite and propyl gallate. Generally, the concentration of antioxidants may range from about 0.02 percent to about 1 percent by weight of the formulation, and preferably in the range from about 0.05 percent to about 0.5 percent by weight of the formulation. Any antioxidants, which are compatible with the present formulation, can be used.

In yet another embodiment the composition may further comprise glycolic acid to increase the absorption efficiency of hydroquinone and assist in the skin lightening effect of the formulation. Preferably the concentration of glycolic acid is not less than about 5.0 percent and not more than about 15.0 percent, particularly preferred concentrations are not less than about 8.0 percent and not more than about 12.0 percent, and most preferably about 10.0 percent.

The formulations of the present invention may further comprise antimicrobial preservatives. Any suitable preservatives can be used in the present composition. Examples of suitable preservatives include for example methyl paraben, propyl paraben and butylated hydroxytoluene.

Formulations of the present invention may further comprise agents that increase the viscosity of the final formulation such as for example xanthum gum.

In another aspect of the invention a less potent composition may be provided as an over-the-counter formulation for reducing wrinkles and skin lightening comprising hydroquinone, retinol, sunscreen and low potency steroid. In one embodiment the composition comprises hydroquinone, tretinoin, sunscreen and a low potency steroid. In a preferred embodiment the composition comprises not more than 2 percent hydroquinone, not more than 0.5 percent retinol a known effective treatment for both wrinkles and skin pigmentation similar to tretinoin, a low potency steroid, preferably class 5 such as hydrocortisone, at a concentration of about 1.0 percent and sunscreen (non-paba) having SPF of not less than about 15 and not more than about 50. In yet another embodiment this composition may further comprise glycolic acid at a concentration of not more than 20 percent.

Preparation and Use

The compositions above may be prepared by mixing to homogeneity the four compounds, hydroquinone, tretinoin, sunscreen and low potency steroid in an amount based on the desired percentage by weight. In addition, an excipient may be added as desired to facilitate use as a cream, as a preservative or as an agent to assist in the uptake of the hydroquinone, tretinoin or low potency steroid. A concentration of glycolic acid may also be added to the formulation to enhance or increase uptake of the active ingredients of the composition or as an additional skin-lightening agent based on the desired percentage by weight.

Once formulated in a cream the composition may be applied by hand to areas of the skin which the user desires lightened by rubbing until the contact between the fingers and the application area are no longer slippery.

One preferred method of making the composition includes boiling and then cooling an appropriate volume of water to 75 degrees C., and dissolving methyl paraben, and sodium metabisulfite to form a first solution; this is followed by cooling the first solution, and adding propylene glycol as an aqueous solution, and hydroquinone, triamcinolone and tretinoin to form a second solution. Next, the following ingredients are combined and heated to 65 degree C. to form a uniform composition Solbar, propyl paraben, xanthum gum, Vitamin E and butyl hydroxytoluene this is added to the second solution to achieve a uniform combined composition.

A preferred composition of the present invention comprises the following active ingredients:

| | | Percentage |
|---|---|---|
| | | (% W/W) |
| hydroquinone | | 8.0 |
| tretinoin | | 0.05 |
| triamcinolone | | 0.025 |
| Solbar | SPF 50 | |

The following compositions was prepared in accordance with the present invention:

| | | | Percentage |
|---|---|---|---|
| | | grams | (% W/W) |
| hydroquinone | | 60.0 | 8.0 |
| triamcinolone | | 0.188 | 0.025 |
| tretinoin | | 0.375 | 0.05 |
| propylene glycol | 100 ml | | |
| methyl paraben | | 0.375 | 0.05 |
| propy paraben | | 0.187 | 0.025 |
| sodium metabisulfite | | 1.50 | 0.20 |
| xanthum gum | | 1.25 | 0.167 |
| vitamin E | 25,000 IU | | |
| butylated hydroxytoluene | | 4.80 | 0.64 |
| Solbar qs | SPF 50 | | |

The gram amounts listed above are for a formulation having a final weight of 750 grams.

We claim:

1. A topical skin lightening cream for the treatment of non-congenital dermal blemishes comprising:
a. hydroquinone wherein said concentration of hydroquinone is not less than 2 percent and not more than 12 percent;
b. tretinoin wherein said concentration of tretinoin is not less than 0.01 percent and not more than 0.10 percent;
c. a steroid wherein said steroid is selected from class 5, 6 or 7 steroids; and
d. sunscreen having a sun protection factor of not less than 15 and not more than 50.

2. A topical skin lightening cream according to claim 1 wherein said concentration of hydroquinone is not less than 4 percent.

3. A topical skin lightening cream according to claim 1 wherein said concentration of hydroquinone is not more than 10 percent.

4. A topical skin lightening cream according to claim 1 wherein said concentration of hydroquinone is 8 percent.

5. A topical skin lightening cream according to claim 1 wherein said concentration of tretinoin is not less than 0.03 percent.

6. A topical skin lightening cream according to claim 1 wherein said concentration of tretinoin is not more than 0.08 percent.

7. A topical skin lightening cream according to claim 1 wherein said concentration of tretinoin is 0.05 percent.

8. A topical skin lightening cream according to claim 1 wherein said steroid is hydrocortisone or triamcilinone.

9. A topical skin lightening cream according to claim 8 wherein said concentration of hydrocortisone is not less than 0.5 percent.

10. A topical skin lightening cream according to claim 8 wherein said concentration of hydrocortisone is not more than 10.0 percent.

11. A topical skin lightening cream according to claim 8 wherein said concentration of hydrocortisone is not less than 1.0 percent.

12. A topical skin lightening cream according to claim 8 wherein said concentration of hydrocortisone is not more than 5.0 percent.

13. A topical skin lightening cream according to claim 8 wherein said concentration of hydrocortisone is 2.5 percent.

14. A topical skin lightening cream according to claim 8 wherein said triamcilinone is not fluorinated.

15. A topical skin lightening cream according to claim 14 wherein said concentration of triamcilinone is not less than 0.01 percent.

16. A topical skin lightening cream according to claim 14 wherein said concentration of triamcilinone is not more than 0.1 percent.

17. A topical skin lightening cream according to claim 14 wherein said concentration of triamcilinone is not less than 0.025 percent.

18. A topical skin lightening cream according to claim 14 wherein said concentration of triamcilinone is not more than 0.05 percent.

19. A topical skin lightening cream according to claim 1 further comprising an excipient.

20. A topical skin lightening cream according to claim 19 wherein said excipient is metabisulfite.

21. A topical skin lightening cream according to claim 1 further comprising glycolic acid.

22. A topical skin lightening cream according to claim 21 wherein the concentration of said glycolic acid is not less than 5.0 percent.

23. A topical skin lightening cream according to claim 21 wherein the concentration of said glycolic acid is not more than 15.0 percent.

24. A topical skin lightening cream according to claim 21 wherein the concentration of said glycolic acid is not less than 8.0 percent.

25. A topical skin lightening cream according to claim 21 wherein the concentration of said glycolic acid is not more than 12.0 percent.

26. A topical skin lightening cream according to claim 21 wherein the concentration of said glycolic acid is 10.0 percent.

27. A topical skin lightening cream for the treatment of non-congenital dermal blemishes comprising:
a. hydroquinone wherein said concentration of hydroquinone is 8 percent;
b. tretinoin wherein said concentration of tretinoin is not less than 0.05 percent;
c. triamcinolone wherein said concentration of triamcinolone is 0.025 percent; and
d. sunscreen having a sun protection factor of 50.

28. A topical skin lightening cream for the treatment of non-congenital dermal blemishes comprising:
a. hydroquinone wherein said concentration of hydroquinone is not more than 2 percent;
b. retinol wherein said concentration of retinol is not more than 0.5 percent;
c. a steroid wherein said steroid is selected from class 5, 6 or 7 steroids; and
d. sunscreen having a sun protection factor of not less than 15 and not more than 50.

29. A topical skin lightening cream according to claim 27 wherein said steroid is a class 5 steroid.

30. A topical skin lightening cream according to claim 30 wherein said steroid is hydrocortisone.

31. A topical skin lightening cream according to claim 31 wherein said concentration of hydrocortisone is 1.0 percent.

32. A topical skin lightening cream according to claim 27 further comprising glycolic acid.

33. A topical skin lightening cream according to claim 33 wherein said concentration of glycolic acid is not more than 20 percent.
